(19) United States
(12) Patent Application Publication
(10) Pub. No.: US 2023/0072205 A1
(43) Pub. Date: Mar. 9, 2023

(54) HEALTH AUXILIARY SYSTEM FOR EVALUATION OF DISEASE RISKS

(71) Applicant: Ever Fortune.AI CO., Ltd., Taichung City (TW)

(72) Inventors: Yu-Cheng LEE, Taichung City (TW); Chien-Hao Huang, Taichung City (TW)

(21) Appl. No.: 17/500,019

(22) Filed: Oct. 13, 2021

(30) Foreign Application Priority Data
Aug. 27, 2021 (TW) 110131867

(51) Int. Cl. G16H 50/30; G16B 30/00; G16H 50/20

(52) U.S. Cl. CPC G16H 50/30; G16B 30/00; G16H 50/20

(57) ABSTRACT

The present invention relates to a health auxiliary system for a user to evaluate disease risks and provide diet recommendations based on the user's physical information and measurement of parameters obtained in the daily routine. The present health auxiliary system includes multiple gene detection units, multiple physical diagnostic unit, a data signal unit, and an asynchronous control signal unit. The use of asynchronous control signal unit can be implemented to reduce power consumption required during computing.

### CROSS-REFERENCE TO RELATED APPLICATIONS

The present application is based on, and claims priority from, Taiwan Patent Application Serial Number 110131867, filed Aug. 27, 2021, the disclosure of which is hereby incorporated by reference herein in its entirety.

### TECHNICAL FIELD

The present invention relates to an auxiliary health system, and more particularly, to an asynchronous controller that controls gene detection data and physical diagnosis data to provide a digital display interval to present a function of evaluating the user's health status in vitro.

### BACKGROUND

Science and technology continue to innovate, and the clinical diagnosis of inspection, auscultation and olfaction, inquiry and palpation has changed to the use of scientific instruments to implement precise clinical diagnosis. The instruments are used to analyze the characteristics of the human body to help users understand their own health conditions. Through comprehensive analysis of the causes and symptoms, the instruments may provide users with suggestions to improve indirectly to their own body constitution including slowing down the aging process by changing diet and strengthening autoimmunity. In the Chinese patent publication CN107194174A (hereinafter referred to as 174'), titled “health condition monitoring method, system and storage media”, it discloses that the use of thirty categories for example including self perception, physical health, sports physical health and mental personality health, age, gender, occupation, height, weight, BMI, blood pressure, blood glucose, blood type, blood lipid, uric acid, body type and the evaluation results of basic body characteristics, sensory system, respiratory system, circulatory system, digestive system, urogenital system, blood system, nervous system, skeletal system, endocrine system, skin system and oral system. The method disclosed in the 174' patent disclosure sequentially includes a first step, which includes the user's health data, a second step, which includes past historical data, and a third step, which includes a human figure to display the evaluation results of the current human health status, and the systems can be printed as a written report. However, the '174 patent disclosure does not explain how to transmit messages through the transmission protocol, which only discloses that operation of the hardware can be form a system through software to be read.

In another previous art, titled “system and method for predicting regimen treatment related results” of R.O.C. patent Publication No. TW201725526A (hereinafter referred to as 526'), it refers to a method applied to toxicity detection for cancer treatment, wherein the method is used to a prediction model of predicting treatment related results and applies it to a plurality of data sets, and a series of machine learning algorithms are applied to the resulting data generated from the application of the prediction model, and the prediction model is used to predict the treatment related results. The 526' patent disclosure does not disclose the actual content of the machine learning algorithm, which only discloses the use of the penalty logistic regression algorithm and the random forest algorithm for the training data set used for the establishment of the prediction model. The 526' patent disclosure belongs to the abstract concept of application layer in “Open System Interconnection Reference Model” formulated by the open network architecture or international organization for Standardization (ISO), it does not disclose how data is handshaking with each other in the physical layer or session layer through the coding of communication protocol.

In yet another previous art, titled “system and program of information exploration data search” of R.O.C. patent No. TW 1696924 (hereinafter referred to as 924'), it discloses that the system and program of exploration data search refer to the photographed spectral data to optimize the verification algorithm, in which the detection algorithm information required of the target event can be identified by searching, and the correlation more than three levels between each target event of the photographed subject and the detection algorithm information are obtained in advance.

### SUMMARY

The problem to be solved by the invention is that when signals of a large number of databases are switched and compared with a large amount of data, scheduling of the signal coding, data path and controller can be performed from the circuit design of the physical layer through the asynchronous circuit design, so that the system operation does not need continuous sampling of clock signals, and thereby achieving the operation mode of power saving or low power. Especially, when a large number of genetic testing data and physical diagnosis data are operated, power consumption has become the main problem of system operation. Especially, when a large number of data are operated by mobile devices, such as mobile phones, tablet computers and notebook computers, the life time of the battery will be greatly reduced due to the extensive use of its system operation.

The method for solving the problem of the invention includes adopting Muller C-element and applying it to the asynchronous controller. After the signals of the asynchronous controller and the data path are collected together, an event signal is generated. The use of the Muller C-element can be a signal meter for the second or third event collecting. After waiting for different times, all of the relevant input signals are collected and then output. Therefore, the use of the element can process and collect different gene detection data and physical diagnosis data, and then transmit a control signal, and generate corresponding output signals to an electronic display device to display different levels of warning prompts.

Compared with the disclosure in the 174' patent, when the health auxiliary identification system of the invention is applied, the difference of color block interval can be presented by spectrum to clearly inform the user of the risk level. Based on the disclosure of 174' patent, it displayed by a human figure, and the user cannot know the different levels of poor health.

In addition, the health auxiliary identification system of the invention can delay the waiting matching of the controller for the gene detection data and physical diagnosis data in operating of the data path through the communication protocol, or even perform the next level operation by the controller when the operation of a data path has not been completed. The 526' patent disclosure only describes the application in cancer treatment toxicology. However, the invention can be applied to beauty and skin care, intestinal bacteria, memory decline, etc. In addition to informing the user of the need for immediate help, the invention uses asynchronous circuits to formulate a communication protocol in different phases, which can achieve the function of power saving or low power consumption.

In addition, when the health auxiliary identification system of the invention is applied, it does not need more than three levels of data correlation as described in 135' patent disclosure. The invention uses an analytic hierarchical process (AHP) operation method to calculate the weight hierarchically and give different priority factors. Therefore, the invention can quickly perform data comparison and operation to produce output, so as to avoid the disadvantage of excessive memory use, and reduce the power consumption of multiple charging and discharging in memory.

### BRIEF DESCRIPTION OF THE DRAWINGS

FIG. 1 shows a block diagram of the invention.

FIG. 2A shows an embodiment of the electronic display device.

FIG. 2B shows an embodiment of the electronic display device.

FIG. 2C shows an embodiment of the electronic display device.

FIG. 3 shows a circuit structure of the connected units in the invention.

FIG. 4 shows a utilization of Muller C-elements with the asynchronous control unit in the invention.

FIG. 5 shows a protocol between two units.

FIG. 6 shows a control signal timing diagram for 2-phase protocol.

FIG. 7 shows a control signal timing diagram for 4-phase protocol.

FIG. 8 illustrates a flowchart of a method of utilizing an asynchronous control unit to create output.

### DETAILED DESCRIPTION

Some preferred embodiments of the present invention will now be described in greater detail. However, it should be recognized that the preferred embodiments of the present invention are provided for illustration rather than limiting the present invention. In addition, the present invention can be practiced in a wide range of other embodiments besides those explicitly described, and the scope of the present invention is not expressly limited except as specified in the accompanying claims.

The health auxiliary identification system of the present invention is described in detail below with reference to the accompanying drawings.

Taking stool analysis for the digestive tract as an example, feces can be used to detect bacteria in the intestines on the NGS platform. The platform is usually used to detect the types and proportions of bacteria resided in the tester's intestines as well as detection on harmful bacteria. The platform can inform the user of the current intestinal status of the test results. On the other hand, the present invention combines information obtained from the analytical platform such as next generation sequencing (NGS) with questionnaires answered by the user to achieve a much accurate prediction or understanding of the premonition regarding to health issues that a tester is facing. Different display methods are incorporated with the genetic test results to demonstrate abnormality, ranges of risks from low to high, for which a user may adjust his or her own daily routine, dieting habit, and sleeping to avoid infections or diseases.

For the example of cosmetology, genetic information received from using platforms would be improving on the skincare. Note that a person may perform a wrinkles detection on the platform and also answer the questionnaires relating to the user's age, and frequency of receiving sun bath. If the result returns by the platform showing the person is genetically in high risk, but the questionnaire result turn out to be low in risk, the finally analysis of the invention may send a signal to warn the person with possible progression of risks in getting wrinkles in the near future. Once the person known about the warning, he or she may decide to use cosmetics or facial masks earlier to slow down the wrinkles from revealing one's ages.

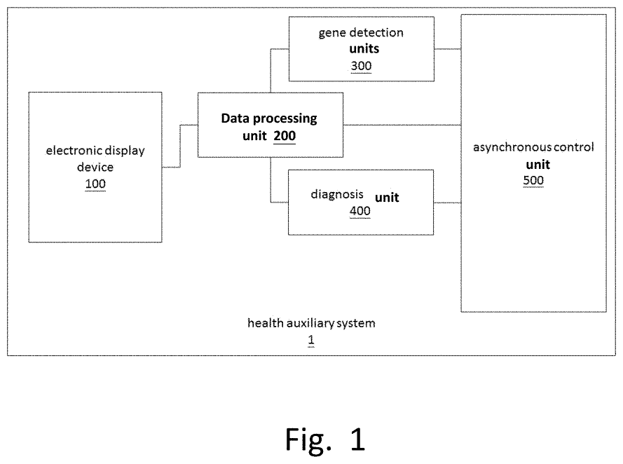

Referring first to FIG. 1, it shows a block diagram of a health auxiliary system 1, which is designed for evaluation of disease risks. The health auxiliary system 1 includes an electronic display device 100, a plurality of gene detection units 300, a plurality of diagnosis units 400, a data processing unit 200 and an asynchronous control unit 500. The electronic display device 100 is coupled to receive signals processed by the data processing unit 200. The data processing unit 200 is coupled to the plurality of gene detection units 300, the plurality of diagnosis units 400 and the asynchronous control unit 500. The asynchronous control unit 500 is coupled to the plurality of gene detection units 300 and the plurality of diagnosis units 400.

Electronic Display Device

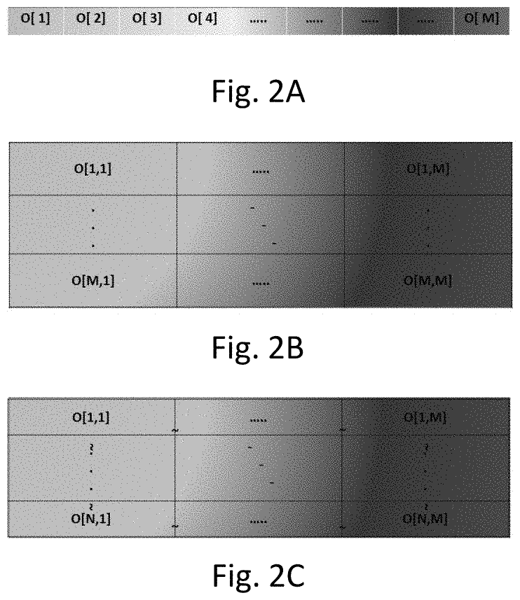

FIG. 2A to 2C show the state of the electronic display device 100. The electronic display device can be arranged in the form of matrix units of NM, NxM or MxM, wherein N is the longitudinal direction of the matrix, M is the transverse direction of the matrix, and N and M are the natural number representation of integers from natural number 1 to infinity. The electronic display device 100 includes light-emitting diodes to display the alarm signal displayed in the matrix according to the gradient change of the visible spectrum of 400 to 700 nm emitted by the light-emitting diodes.

Taking FIG. 2A as an example, it shows a matrix of NM, in which there are M levels of light spot matrix in one row. If the risk level can be presented in the first LED element O [1] to the fourth LED element O [4] by 1×4, it indicates normal, attention, danger and ultra danger in sequence, and displays the level of early warning to be notified by the health auxiliary identification system through the color of the LEDs according to the visible spectrum of 400 to 700 nm. For example, green is normal; and the more red it is, the more dangerous it is.

In another embodiment of the electronic display device 100 shown in FIG. 2B, the size of the matrix for the electronic display device 100 is shown as a square matrix with equal number of rows and columns. In a preferable embodiment, the size of the square matrix could be 2-by-2, 3-by-3, or M-by-M. Furthermore, the matrix for the electronic display device is divided into several cells, and each cell may be representing different aspects of factors in the warning system. For example, in a 3-by-3 matrix, the vertical axis of the electronic display device may be represented as the physical condition of the user, and the arrangement of the representation includes not urgent (low degree of urgency for improvement), urgent, and very urgent for physical condition check-up. Also, the horizontal axis may be represented as the risks including the first (low) degree risk, second (middle) degree risk, and third (high) degree risk. The arrangement for the corresponding physical condition versus risks is included but not limited to the example shown in FIG. 2B. The arrangement can also be from high risk to low risk. In FIG. 2B of the 3-by-3 matrix, O [1,1] is shown in color of green to indicate a safe signal, and O [3,3] is shown in red as a signal to represent an extreme danger.

FIG. 2C is another embodiment of the electronic display device 100, in which the matrix may be arranged in a N-by-M rectangular. The number of the row is set to be less than the number of the column or the number of the column is less than the number of the row. The coloration with gradient distribution for each cell in the matrix may be arranged to display. In FIG. 2C, the N-by-M matrix could have the signals converted into Fourier Transform to show the change in the signal spectrum and to display the warning signals with a real-time dynamic change which is similar to the MIDI equalizer used for adjustment on the volume of the sound.

Gene Detection Units

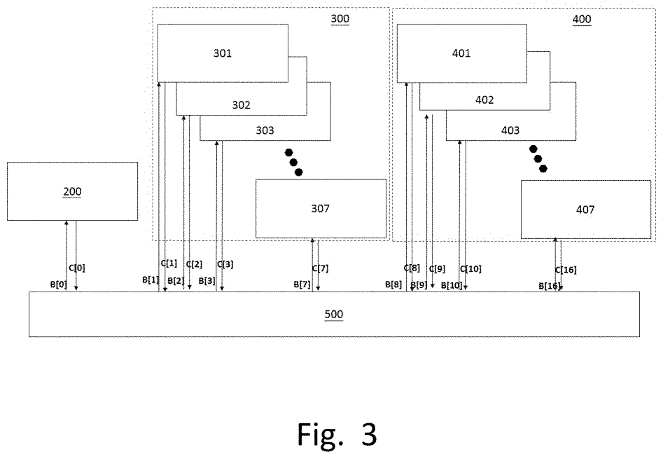

Human chromosomes are composed of proteins and genes, and the genes consist of four nucleic acid bases; they are adenine (A), cytosine (C), guanine (G), and thymine (T). We take the bases as genetic signal fed in the circuit for applications. These nucleic acid bases in DNA could be formed in different combination and a lengthy arrangement or known as a sequence. The order of sequence with these four bases determines the factors in the human genetic codes for human diseases, growth conditions, aging conditions, and so on. FIG. 3 shows the structure of the units of the present invention. Among them, the gene detection unit 300 includes a plurality of gene datasets 301 to 307, and these gene databases correspond to the segment of genetic sequence on the aforementioned growth conditions, daily routine and application, drug allergy, and detection of diseases, and the plurality of gene dataset (301 to 307) can be historical data that contains a particular ethnic group or the continuous growth of an individual in the past as well.

The example of gene dataset is based on historical data on the growth of human race. The first genetic testing dataset 301 is the gene dataset of the Austronesian population. The second genetic testing dataset 302 is the gene dataset of the Han population. The third genetic testing dataset 303 is the gene dataset of the Hakka population. The fourth genetic testing dataset 304 is the gene dataset of the Vietnamese population. The fifth genetic testing dataset 305 is the gene dataset of the Thai population. The sixth genetic testing dataset 306 is the gene dataset of the American-born Chinese population. The seventh genetic testing dataset 307 is the gene dataset of Japanese-born Chinese population.

Another example of gene dataset is based on continuous growth of individual. The first genetic testing dataset 301 is the gene dataset of neonatal growth. The second genetic testing dataset 302 is the gene dataset of the growth in infancy. The third genetic testing dataset 303 is the gene dataset of the childhood growth. The fourth genetic testing dataset 304 is the gene dataset of the growth of adolescents under the age of 16. The fifth genetic testing dataset 305 is the gene dataset of the adult growth. The sixth genetic testing dataset 306 is the gene dataset of middle aged growth. The seventh genetic testing dataset 307 is the gene dataset of elderly growth.

The gene detection unit 300 includes genetic testing datasets with flexibility and allows software updates and re-configuration including but not limiting to dataset that is collected for the purpose of recording the growth condition, daily routine, drug allergy, and detection of diseases, and so on.

The body constitution diagnosis unit 400 is depicted in FIG. 3. The diagnosis unit 400 includes a plurality of diagnostic arrays 401 to 407. The first diagnostic array 401 is basic physiological data of a user that includes height, weight, BMI. The second diagnostic array 402 is the data of the 5-D itch scale answered by the user. The third diagnostic array 403 is the data of the Barthel Index answered by the user. The fourth diagnostic array 404 is the user input image evaluation data processed by artificial intelligence, which could be an electrocardiogram (ECG/EKG), a PET scanned image, or a CT scanned image. The fifth diagnostic array 405 is the ELISA data of cortisol (saliva). The sixth diagnostic array 406 is the urine test data. The seventh diagnostic array 407 is the intestinal test data.

In addition, data on the first to the seventh diagnostic arrays can be divided into multiple levels, and its preferred number of levels is three. The advantages is that the data processing unit 200 can speed up the circuit computation in the auxiliary health system 1 and react to generate an accurate average result of about 95.57% to 99.35% during calculation. The gene detection unit 300 uses algorithm of Analytic Hierarchy Process (AHP) in sequence to obtain the consistency matrix (Aν) and the eigenvector (λ), and further obtain the eigenvalue from the eigenvector. Furthermore, a test is done to calculate accepted value, and then locate the appropriate location in the risk matrix.

Next, referring to FIG. 3, the data processing unit 200 reads data from the gene detection unit 300 and the diagnosis unit 400, and compute by a pre-determined statistical formula such as AHP to generate priority weights of different dimensions. The dimensions may be the risk assessment of congenital healthy constitution and acquired constitution, or the recommended intake of supplements based on congenital healthy constitution and acquired constitution.

Referring to FIG. 3, the asynchronous control unit 500 transmits control signals and data signals to the data processing unit 200 bi-directionally via the control bus B[0], data path bus C[0]. The asynchronous control unit 500 also transmits control signals and data signals to the genetic testing datasets (301 to 307) of the gene detection unit 300 via the control bus B[1] to B[7], and the data path bus C[1] to C[7]. Similarly, the asynchronous control unit 500 transmits control signals to the diagnostic arrays (401 to 407) of the diagnosis unit 400 via the control bus B[8] to B[16], and the data path bus C[8] to C[16].

Algorithm of AHP

The asynchronous control unit 500 reads control signals according to the user's options of request to predict disease outcomes, growth progress, and aging condition, and so on by selecting the appropriate criteria of dataset from the gene detection unit 300 through the data processing unit 200 using analytic hierarchical process (AHP) method. The AHP is adopted to receive priority matrix for determining the SNP or the genomes as the priority when seeking solutions for the interested disease outcomes, growth progress, and aging condition, and so on. After the AHP is performed, the data processing unit 200 is able to provide a constant, with a range of 1 to 7, to indicate a priority when accessing the dataset for the preferred gene detection unit 300. Meanwhile, the data processing unit 200 matches the priority in weight to the diagnostic array within the selected the diagnosis unit 400. The expression of the cells in the priority matrix as the weight to the preferred solutions is noted in Equation [1], where

Aᵢⱼ=1/aᵢⱼ Equation [1]

The cell locations are diagonally symmetrical with value of 1 along the diagonal cells. The rest of cells within the priority matrix A is expressed as Equation [1], and the values are reciprocal as position of row and column are reversed. For example, if A₁₂=2, then a₂₁=½. The priority matrix A would be A=[1 2; ½ 1].

After obtaining the priority matrix A, all the cells in the column are added up and each of the cells in the priority matrix A is divided by the sum of the cells in the added-up column to perform standardization. Then, each row of the matrix A is added up horizontally in order to obtain eigenvector (w). The eigenvector is also the priority vector. The priority vector is introduced into the priority matrix to obtain λₘₐₓ to verify that the value passes Satty's test and to assure the region of confidence is consistent. According to the health-related criteria to be provided and the relevant weight of the risk level, the relevant proportion matrix between the factors at all levels in the hierarchical analysis method can be performed again, and the standardized matrix can be established to calculate the relevant weight value and consistency verification of each risk assessment level until the weight calculation of the factors at all levels is completed.

Circuit Operation

Referring to FIG. 3, the circuit of the health auxiliary system is set to have the controller that receives signals from data path and send out next control signals in the next output state. The circuit is mainly divided by data path and control circuit path. That is, the asynchronous control unit 500 gathers the control signals from control buses B[0] to B[16] and signals from data buses C[0] to C[16] together prior to output next state control signals. The asynchronous control unit 500 is configured with an asynchronous self-timed state machine. For each state, the control signals are coded from signals generated from the gene detection unit 300 and the diagnosis unit 400 to form a 16-bit long control signals (state coding). The asynchronous control unit 500 reads the signals from the gene detection unit 300 and the diagnosis unit 400 as a current state and then combine an outputting signal (value) for the next state. The truth table is illustrated and provided in TABLE 1, where signal is abbreviated as “Sg”, and state is abbreviated as “St”. The circuitry of the asynchronous control unit 500 may be computer generated by entering the truth table and synthesis the hardware description language in behavioral way or structural way by VHDL or Verilog.

TABLE 1
Truth table for asynchronous control unit
Current State

| Sg | G | G | G | G | G | G | G | G | H | H | H | H | H | H | H | H |
|---|---|---|---|---|---|---|---|---|---|---|---|---|---|---|---|---|
| St | 1 | 2 | 3 | 4 | 5 | 6 | 7 | 8 | 1 | 2 | 3 | 4 | 5 | 6 | 7 | 8 |
| S0 | 0 | 0 | 0 | 0 | 0 | 0 | 0 | 0 | 0 | 0 | 0 | 0 | 0 | 0 | 0 | 0 |
| S1 | 0 | 0 | 0 | 0 | 0 | 1 | 0 | 0 | 0 | 0 | 0 | 0 | 0 | 0 | 1 | 0 |
| S2 | 0 | 0 | 0 | 0 | 1 | 0 | 0 | 0 | 0 | 0 | 0 | 0 | 0 | 1 | 1 | 0 |

Next State

| Sg | G | G | G | G | G | G | G | G | H | H | H | H | H | H | H | H |
|---|---|---|---|---|---|---|---|---|---|---|---|---|---|---|---|---|
| St | 1 | 2 | 3 | 4 | 5 | 6 | 7 | 8 | 1 | 2 | 3 | 4 | 5 | 6 | 7 | 8 |
| S1 | 0 | 0 | 0 | 0 | 0 | 1 | 0 | 0 | 0 | 0 | 0 | 0 | 0 | 0 | 1 | 0 |
| S2 | 0 | 0 | 0 | 0 | 1 | 0 | 0 | 0 | 0 | 0 | 0 | 0 | 0 | 1 | 1 | 0 |
| S5 | 0 | 0 | 0 | 0 | 1 | 1 | 1 | 0 | 0 | 0 | 0 | 1 | 0 | 1 | 1 | 0 |

Muller C-Element

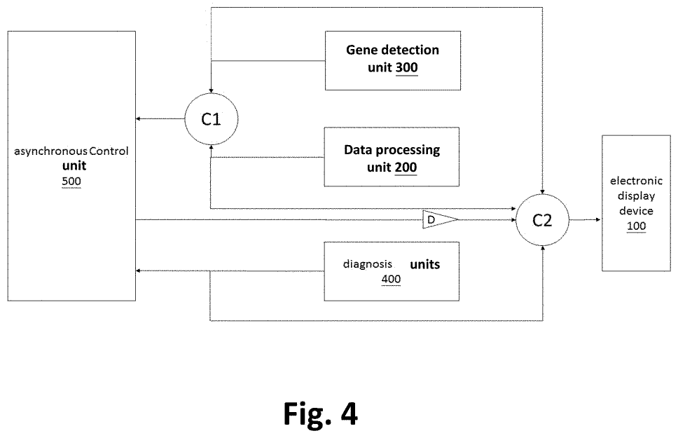

Referring to FIG. 4. Muller C-element is used in the design of asynchronous control unit 500. The controller signals in the asynchronous control unit 500 usually output faster than in the data path circuit, therefore Muller C-element, an event logic including several and-gate, is used for the collection of all required signals. In FIG. 4, the first Muller C-element (C1) gather complete set of signals from the gene detection unit 300 by a gene detection unit reading step S11 and signals from data processing unit 200 in the data-signal-unit reading step S21. In the genetic-detection-unit reading step S11, the asynchronous control unit 500 reads data signals from the gene detection unit 300, data processing unit 200, and the firing signal (output signal from the first Muller C-element) of gene detection unit 300 and data processing unit 200. In the genetic-detection-unit reading step S21, the step allows the gene detection databases (301-307) within the data processing unit 200 to calculate the priority of the gene detection database by using the analytical hierarchical analysis method. The gene detection unit reading step S11 and a diagnostic-unit reading step S21 are coded with 16-bit in the control buses for the maximum of states allowed and also the intents of Hex conversion for fast manipulation of big ending or little ending in the control signals. Next, the pre-determined AHP result is stored in a lookup-table, in which data processing unit 200 takes a lookup-table comparing step S31 by comparing the data in the genetic detection database 301-307 and the signals received from the genetic detection unit 300 and outputs genes information with prioritized sequence signals. Next, the asynchronous control unit 500 takes signal reading from the first Muller C element (C1) and diagnosis unit 400 as the diagnostic-unit reading step S41 for a pre-determined control signal for next state in the asynchronous control unit 500. The asynchronous control unit 500 then takes an asynchronous controlling step, in which it matches the time required to a corresponding delay element (D) before outputting the signal to the second Muller C-element (C2). The use of the delay element (D) and the use of the Muller C-element (C2) are referred as a data path delay controlling step S51, which is used to control the event of waiting all controlling signals before sending a firing signal to the Muller C-element. Upon the signal arrival generated control signal from the previous state and the data signals, the asynchronous control unit 500 triggers state machine and send signal to Muller-C element or referred as an asynchronous controlling step S61. Finally, an outputting display step S71 is executed and the result of a risk assessment shown on electronic display device 100. The execution of the outputting display step S71 also involves the use of gates such as AND, OR, and NOT to further control the output signals on the designated location to allow the LED display in the matrix formation as shown by the electronic display device 100.

Asynchronous Control Signals and Protocol

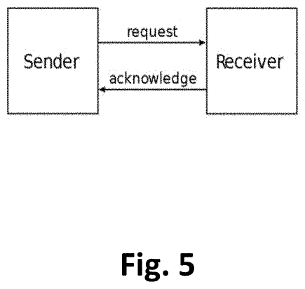

Referring to FIG. 5, the signal transmission between the units, particularly between the control unit and the data unit, requires a communication protocol to determine the timing (time sequence) and data transmission. For example, a unit 1 sends a request signal (request) to the unit 2 and trigger unit 2 to send a signal back. That is, after unit 2 receives the request signal, it returns an acknowledgement signal (acknowledge), and then the data transmission becomes feasible.

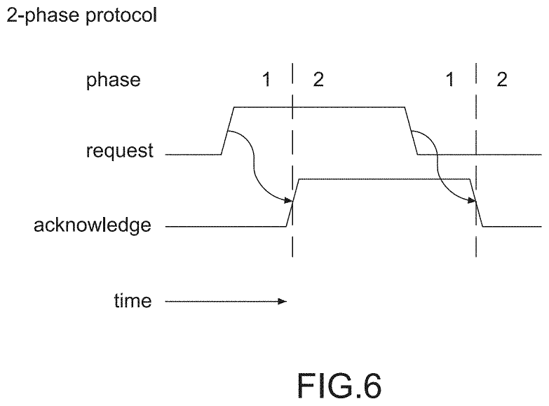

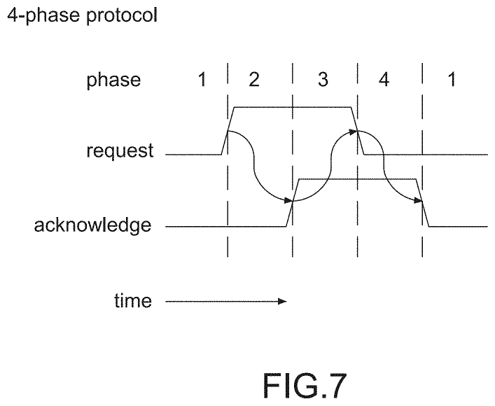

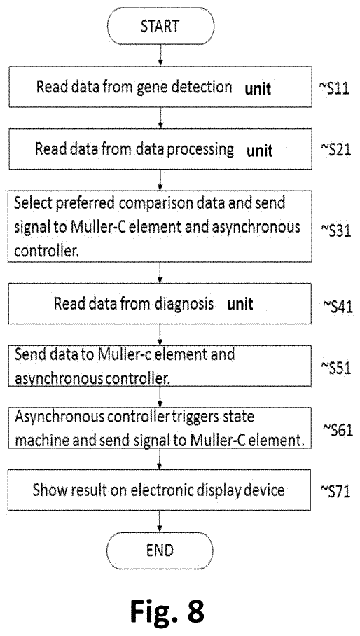

The communication protocol of the asynchronous control methodology can be classified into two-phase (2-phase) and four-phase (4-phase). FIG. 6 is a two-phase transmission method, and FIG. 7 is a four-phase asynchronous communication protocol. The difference between the two-phase and four-phase communication protocols is the coding of the control signal used to decide state of the control signal when initiating data transmission.

Referring to FIG. 6, the timing diagram shows that after the request signal (request) switches from 0 to 1, the acknowledge signal (acknowledge) also switches from 0 to 1; and the request signal must be switched from 1 to 0 after the acknowledge signal is activated. The acknowledge signal must also be switched off when the request signal is off. From observing the communication protocol with a coding aspect, the request signal (request) and the acknowledge signal (acknowledge) can be regarded as a phase of (0,1) or (1,0), and (1,1) or (0,0) as another phase. Thus, it is called two-phase.

Referring to FIG. 7, a four-phase communication protocol is presented in the timing diagram. The coding of the communication protocol regards the request signal (request) and the acknowledge signal (acknowledge) as two signal cables, and the coding status of the signal pairs are (1,0), (1,1), (0,1), and (0,0) to be distinguished as four different states. For implementation of software encoding, the control signals are much easier than the hardware implementation, and the hardware implementation can also be achieved through a combination of AND, OR, and NOT gates with the help of tools on hardware description language.

Moreover, the data path circuit usually consumes larger gate counts than the asynchronous control circuit in the synthesis of the hardware design. Therefore, the asynchronous control circuit must have added a delay circuit or known as delay element to match the time required for the data path computation and to initiate the next-state control signal correctly. Unlike the synchronous control circuit, which samples the clock signal to obtain the correct calculation value, the rise and fall of the control signals actually consumes lots of power consumption, and which is a disadvantage to the use of mobile devices. Conventionally, flip-flops are used, but we have implemented the present invention by adding a delay circuit to the asynchronous control circuit.

As a domain of asynchronous controller used in the health risk assessment, hardware design provides an opportunity to design an assessment system with low power consumption and faster protocol than the software implementation. The method for evaluating disease risks of the invention comprises a genetic-detection-unit reading step S11 to read signals from a plurality of genetic detection databases within the gene detection unit; a data-signal-unit reading step S21 that reads signals from the data signal unit to calculate the priority of the genetic detection database by using a hierarchical analysis method; a lookup-table comparing step S31 that compares the genetic detection database and signals received from the genetic unit and outputs with prioritized genes information; a diagnostic-unit reading step S41; an asynchronous controlling step matches the time required to a corresponding delay element before outputting the signal; a data path delay controlling step S51; an asynchronous controlling step S61, and an outputting display step S71. The steps of present invention can be implemented in the circuit design, and applied to portable devices. The invention provides advantages on the use of memories, the reduction of unnecessary charging and discharging of the clock signals by event-driven, and to achieve low power consumption.

As will be understood by persons skilled in the art, the foregoing preferred embodiment of the present invention illustrates the present invention rather than limiting the present invention. Having described the invention in connection with a preferred embodiment, modifications will be suggested to those skilled in the art. Thus, the invention is not to be limited to this embodiment, but rather the invention is intended to cover various modifications and similar arrangements included within the spirit and scope of the appended claims, the scope of which should be accorded the broadest interpretation, thereby encompassing all such modifications and similar structures. While the preferred embodiment of the invention has been illustrated and described, it will be appreciated that various changes can be made without departing from the spirit and scope of the invention.

What is claimed is:

1. A health auxiliary system for evaluation of disease risks, comprising:
an asynchronous control unit;
a plurality of gene detection units coupled to said asynchronous control unit;
a plurality of diagnosis units coupled to said asynchronous control unit;
a data processing unit coupled to said asynchronous control unit; and
an electronic display device coupled to said data processing unit, wherein said electronic display device receives signals processed by the data processing unit.

2. The system of claim 1, wherein said electronic display device is arranged in a matrix.

3. The system of claim 1, wherein said plurality of gene detection unit includes a plurality of gene datasets.

4. The system of claim 1, wherein signals of said plurality of gene detection units, said plurality of diagnosis units and said data processing unit are operated by a two-phase protocol.

5. The system of claim 1, wherein signals of said plurality of gene detection units, said plurality of diagnosis units and said data processing unit are operated by a four-phase protocol.

6. The system of claim 1, wherein said asynchronous control unit includes a first Muller C-element.

7. The system of claim 6, wherein said asynchronous control unit further includes a second Muller C-element.

8. The system of claim 1, wherein said data processing unit coupled to said plurality of gene detection units.

9. The system of claim 1, wherein said data processing unit coupled to said plurality of diagnosis units.

10. The system of claim 9, wherein each of said plurality of diagnosis units is a body constitution diagnosis unit.

11. A method for evaluating disease risks, comprising:
reading data signals by an asynchronous control unit from a plurality of gene detection units;
determining a priority of a plurality of gene detection databases by a data processing unit using a hierarchical analysis method;
comparing data in said plurality of gene detection database and signals received from said gene detection unit and outputs genes information with prioritized sequence signals;
reading control signals for next state in said asynchronous control unit by a plurality of diagnostic units;
matching a time required to a corresponding delay element by said asynchronous control unit before outputting a signal; and
performing a data path delay controlling by said corresponding delay element.

12. The method of claim 11, wherein said corresponding delay element is used to control an event of waiting all controlling signals before sending a firing signal to a Muller C-element.

13. The method of claim 12, further comprising an asynchronous controlling including a first stage and a second stage, wherein said first stage is a two-phase protocol for controlling events logics signals of said gene detection unit and the data processing unit, and said second stage is a four-phase protocol for controlling signals from said data processing unit, said diagnosis unit and said event logic signals collected by said Muller C-element.

14. The method of claim 13, wherein signals of said plurality of gene detection units, said plurality of diagnosis units and said data processing unit are operated by said two-phase protocol.

15. The method of claim 13, wherein signals of said plurality of gene detection units, said plurality of diagnosis units and said data processing unit are operated by a four-phase protocol.

16. The method of claim 11, wherein said asynchronous control unit is coupled to said plurality of gene detection units, said plurality of diagnosis units and said data processing unit.

17. The method of claim 11, wherein said asynchronous control unit further includes a second Muller C-element.

18. The method of claim 11, wherein said data processing unit coupled to said plurality of gene detection units.

19. The method of claim 11, wherein said data processing unit coupled to said plurality of diagnosis units.

20. The method of claim 11, wherein said outputting a signal as a result of a risk assessment is shown on an electronic display device.
